(19) United States
(12) Patent Application Publication
(10) Pub. No.: US 2024/0122938 A1
(43) Pub. Date: Apr. 18, 2024

(54) METHODS AND COMPOSITIONS FOR TREATING UVEAL MELANOMA

(71) Applicants: INSERM (INSTITUT NATIONAL DE LA SANTÉ ET DE LA RECHERCHE MÉDICALE), Paris (FR); UNIVERSITE COTE D'AZUR, Nice (FR)

(72) Inventors: Robert BALLOTTI, Nice Cedex 3 (FR); Corine BERTOLOTTO-BALLOTTI, Nice Cedex 3 (FR)

(21) Appl. No.: 17/768,675

(22) PCT Filed: Oct. 28, 2020
(86) PCT No.: PCT/EP2020/080285

§ 371 Date: Apr. 13, 2022

(30) Foreign Application Priority Data
Oct. 29, 2019 (EP) 19205966.5

(51) Int. Cl. A61K 31/5377; A61K 31/519; A61P 35/00

(52) U.S. Cl. CPC A61K 31/5377; A61K 31/519; A61P 35/00

(57) ABSTRACT

The present invention relates to a compound ABT-263 or a derivative thereof for use in the treatment of uveal melanoma and/or uveal melanoma resistant. Inventors provide evidence that ABT-263 displays clear antiproliferative and proapoptotic activities in metastatic uveal melanoma cells both in vitro and in vivo. They also demonstrated that ABT-263 effect is accompanied with the activation of the ER stress response pathway that exerts cytoprotective effect. Blocking ER stress enhanced ABT-263 killing efficacy. The combination of ABT-263 with PERK inhibition synergistically reduced the survival rate of primary uveal melanoma cells.

### FIELD OF THE INVENTION

The present invention is in the field of medicine, and in particular in the field of oncology. More particularly, the invention relates to methods and compositions for treating uveal melanoma.

### BACKGROUND OF THE INVENTION

Uveal melanoma is the most common primary intraocular malignancy in adult population. It develops de novo from eye's melanocytes, more rarely from a pre-existing nevus. At diagnosis, only 1-3% of the patients have detectable metastases, but it seems that micrometastases can be established several years before the diagnosis of melanoma. Despite enucleation or radiotherapy of the primary lesion, metastases develop in 50% of patients and clinical outcomes are dismal. In 80-90% of cases, the liver is the first metastatic site.

Defective apoptosis, which contribute to sustained cell survival, is a major causative factor in the development and progression of cancer. The ability of a cell to undergo apoptosis is governed by pro- (e.g., BAX, BID, BIM, NOXA) and anti-apoptotic (e.g., BCL-2, BCL-XL, MCL-1) members of the BCL-2 protein family¹.

BCL-2 exerts its antiapoptotic functions by modulating the mitochondrial release of cytochrome c and the interaction of apoptosis activating factors with caspase 9 and BAX (Bcl-2 associated X protein).

Overexpression of the pro-survival BCL-2 family members is commonly associated with cancer². In uveal melanomas, expression of BCL-2, the very pro-survival gene, was shown to be significantly higher compared with normal uveal melanocytes^{1,3}. Such abnormalities can be exploited by chemotherapeutic strategies, such as the BH3-mimetic drugs, to counteract the apoptotic blocks, and halt tumor progression^{4,5}. Several BH3 mimetics, including ABT-737, ABT-263 (Navitoclax) and ABT-199 (Venetoclax) have been developped⁶. ABT-263 (Navitoclax), an orally available derivate of ABT-737, was tested as a single agent in phase I/II for the treatment of different solid and haematological cancers, yet side effects such as thrombocytopenia have been reported^{5,7,8}. ABT-199 is an orally bioavailable, second-generation BH3 mimetic that inhibits BCL-2, that displays much less activity against BCL-xL. Further, ABT-199 exhibits much less side effects, becoming the first BH3 mimetic drug approved by the US Food and Drug Administration for the treatment of some leukemias and lymphomas⁹.

Uveal melanoma metastases are remarkably refractory to conventional chemotherapies and non-sensitive to external radiotherapy. Currently, there are no approved systemic treatments for uveal melanoma once it has spread. 90% of patients will die within 6 months after diagnosis of metastases. This highlights an urgent need for efficient therapeutics improving patients' survival.

### SUMMARY OF THE INVENTION

The invention relates to a compound ABT-263 or a derivative thereof for use in the treatment of uveal melanoma. In particular, the invention is defined by claims.

### DETAILED DESCRIPTION OF THE INVENTION

For the first time, inventors provide evidence that ABT-263 displays clear antiproliferative and proapoptotic activities in metastatic uveal melanoma cells both in vitro and in vivo. They also demonstrated that ABT-263 effect is accompanied with the activation of the ER stress response pathway that exerts cytoprotective effect. Blocking ER stress enhanced ABT-263 killing efficacy. The combination of ABT-263 with PERK inhibition synergistically reduced the survival rate of primary uveal melanoma cells that are more resistant to ABT-263's effect than the metastatic cells.

Accordingly, in a first aspect, the invention relates to a compound ABT-263 or a derivative thereof for use in the treatment of uveal melanoma.

In a particular embodiment, the invention relates to a method for treating uveal melanoma in a subject in need thereof comprising a step of administering to said subject a therapeutically effective amount of compound ABT-263 or a derivative thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. In a particular embodiment, the subject is human. Particularly, in the present invention, the subject has or is susceptible to have uveal melanoma. Particularly, in the present invention, the subject has or is susceptible to have primary uveal melanoma. More particularly, in the present invention, the subject has or is susceptible to have uveal melanoma resistant.

As used herein, the term “compound ABT-263” also known as Navitoclax refers to a synthetic small molecule and an antagonist of a subset of the B-cell leukemia 2 (Bcl-2) family of proteins with potential antineoplastic activity. It has the following chemical formula and structure (Formula I): CAS number: 923564-51-6; C47H55ClF3N5O6S3, 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]-benzamide:

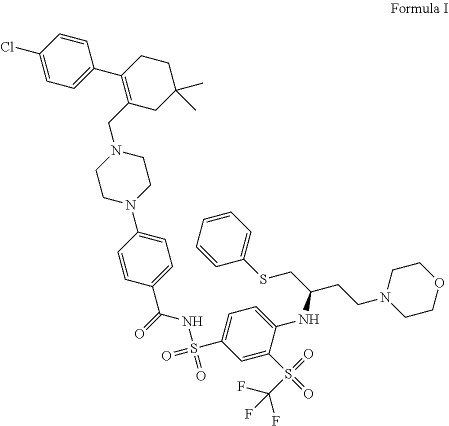 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

As used herein, the term “uveal melanoma” (UM) refers to cancer (melanoma) of the eye involving the iris, ciliary body, or choroid (collectively referred to as the uvea). It is the most common primary intraocular malignancy in adult population. It develops de novo from eye's melanocytes, more rarely from a pre-existing nevus. Half of UM patients succumb to the effects of metastatic disease. Once UM is metastatic, the median survival for patients is only 3-6 months.

In a second aspect, the invention relates to a compound ABT-263 for use in the treatment of metastatic uveal melanoma.

In a particular embodiment, the invention relates to a compound ABT-263 for use in the treatment of uveal melanoma resistant.

As used herein, the terms “uveal melanoma resistant” or “metastatic uveal melanoma” refer to a uveal melanoma which does not respond to a classical treatment such as immunotherapy, protontherapy or radiotherapy. The uveal melanoma may be resistant at the beginning of treatment or it may become resistant during treatment. The resistance to drug leads to rapid progression of metastatic of uveal melanoma. Uveal melanoma metastases are remarkably refractory to conventional chemotherapies and non-sensitive to external radiotherapy.

In a particular embodiment, the uveal melanoma is resistant to conventional therapies: radiotherapy, chemotherapy, protonotherapy, targeted treatment and/or immunotherapy.

In a particular embodiment, the uveal melanoma is resistant to conventional therapies (radiotherapy, chemotherapy, immunotherapy etc).

In a particular embodiment, the metastatic uveal melanoma is resistant to radiotherapy.

As used herein, the term “radiation therapy” or “radiotherapy” have their general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In a particular embodiment, the metastatic uveal melanoma is resistant to chemotherapy.

As used herein, the term “chemotherapy” refers to use of chemotherapeutic agents to treat a subject. As used herein, the term “chemotherapeutic agent” refers to chemical compounds that are effective in inhibiting tumor growth.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In a particular embodiment, the metastatic uveal melanoma is resistant to protontherapy.

As used herein, the term “proton therapy” or “proton radiotherapy” refers to a type of particle therapy that uses a beam of protons to irradiate diseased tissue, most often in the treatment of cancer. The chief advantage of proton therapy over other types of external beam radiotherapy is that as a charged particle the dose is deposited over a narrow range of depth, and there is minimal entry, exit, or scattered radiation dose.

In a particular embodiment, the metastatic uveal melanoma is resistant to immunotherapy.

As used herein, the term “immunotherapy” refers to the use of the compounds which modulate the immune system.

Typically, the following types of immunotherapy can be used to treat melanoma: monoclonal antibodies, immune checkpoint inhibitors or cancer vaccines. In a particular embodiment, the method according to the invention is suitable to predict the risk of relapse to the treatment with immune checkpoint inhibitors. As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In a further embodiment, the uveal melanoma is resistant to a treatment with an immune checkpoint inhibitor.

In a further embodiment, the metastatic uveal melanoma is resistant to a treatment with an immune checkpoint inhibitor.

As used herein, the term “immune checkpoint inhibitor” refers to any compound inhibiting the function of an immune checkpoint protein. Inhibition includes reduction of function and full blockade. In some embodiments, the immune checkpoint inhibitor is an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-éthyl]amino}-1,2,5-oxadiazole-3 carboximidamide:

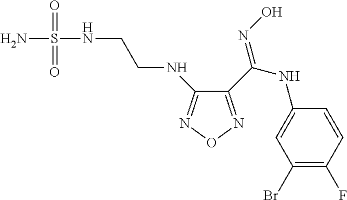 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

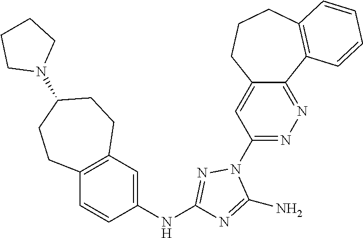 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In a further embodiment, the uveal melanoma is resistant to a targeted treatment.

In a further embodiment, the metastatic uveal melanoma is resistant to a targeted treatment.

As used herein, the term “targeted treatment” refers to treatments used to treat melanoma cutaneous in the art. Typically, targeted treatment refers to use of inhibitors of following genes having mutations (single or double) BRAF, MEK or NRAS. The resistance can be also caused by a double-negative BRAF and NRAS mutation.

In a particular embodiment, the uveal melanoma is resistant to a treatment with the inhibitors of BRAF mutations. In a particular embodiment, the metastatic uveal melanoma is resistant to a treatment with the inhibitors of BRAF mutations.

BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E.

The inhibitors of BRAF mutations are well known in the art. In a particular embodiment, the melanoma is resistant to a treatment with vemurafenib. Vemurafenib also known as PLX4032, RG7204 ou RO5185426 and commercialized by Roche as Zelboraf. In a particular embodiment, the melanoma is resistant to a treatment with dacarbazine. Dacarbazine also known as imidazole carboxamide is commercialized as DTIC-Dome by Bayer. In a particular embodiment, the melanoma is resistant to a treatment with dabrafenib also known as tafinlar which is commercialized by Novartis.

In a particular embodiment, the uveal melanoma is resistant to a treatment with the inhibitors of MEK mutations. In a particular embodiment, the metastatic uveal melanoma is resistant to a treatment with the inhibitors of MEK mutations.

As used herein, the term “MEK” refers to Mitogen-activated protein kinase kinase, also known as MAP2K, MEK, MAPKK. It is a kinase enzyme which phosphorylates mitogen-activated protein kinase (MAPK). MEK is activated in melanoma. The inhibitors of MEK are well known in the art. In a particular embodiment, the melanoma is resistant to a treatment with trametinib also known as mekinist which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, the uveal melanoma is resistant to a treatment with the inhibitors of NRAS mutations. In a particular embodiment, the metastatic uveal melanoma is resistant to a treatment with the inhibitors of NRAS mutations.

The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations. In a particular embodiment, the melanoma is resistant in which double-negative BRAF and NRAS mutant melanoma.

In a particular embodiment, the uveal melanoma is resistant to a combined treatment. In a particular embodiment, the metastatic uveal melanoma is resistant to a combined treatment.

As used herein, the terms “combined treatment”, “combined therapy” or “therapy combination” refer to a treatment that uses more than one medication. The combined therapy may be dual therapy or bi-therapy. In the context of the invention, the uveal melanoma is resistant to a combined treatment characterized by using an inhibitor of BRAF mutation and an inhibitor of MEK as described above. For example, the combined treatment may be a combination of vemurafenib and cotellic.

### Combined Preparation

Inventors have shown that ABT-263 causes endoplasmic reticulum (ER) stress and activate the unfolded protein response (UPR) in primary melanoma cells, whereas in metastatic cells, ABT-263 elicited partial activation of the UPR. The combination of ABT-263 with PERK inhibition synergistically reduced the survival rate of primary uveal melanoma cells. Therefore, inhibition of anti-apoptotic BCL-2 proteins by ABT-263 or a derivative thereof alone or in combination with ER stress inhibitor represents a valid therapeutic strategy for the treatment of uveal melanoma cells.

Accordingly, in a second aspect, the invention relates to a i) compound ABT-263 or a derivative thereof, and ii) an endoplasmic reticulum (ER) stress inhibitor, as a combined preparation for simultaneous, separate or sequential use in the treatment of uveal melanoma.

In another embodiment, the invention relates to a i) compound ABT-263 or a derivative thereof, and ii) an endoplasmic reticulum (ER) stress inhibitor, as a combined preparation for simultaneous, separate or sequential use in the treatment of primary uveal melanoma.

In another embodiment, the invention relates to a i) compound ABT-263 or a derivative thereof, and ii) an endoplasmic reticulum (ER) stress inhibitor, as a combined preparation for simultaneous, separate or sequential use in the treatment of metastatic uveal melanoma and/or uveal melanoma resistant.

In a particular embodiment, the combined preparation according to the invention, wherein ER stress inhibitor is PERK, CHOP or IRE1α inhibitor.

In a particular embodiment, the metastatic uveal melanoma and/or uveal melanoma are resistant to radiotherapy, protonotherapy, chemotherapy, immunotherapy and/or targeted therapy as defined above.

As used herein, the term “endoplasmic reticulum (ER)” is both a major intracellular calcium store and the place where proteins entering the secretory pathway are synthesized, folded, modified, and delivered to their final cell surface or extracellular destination.

As used herein, the term “ER stress” refers to a disturbance in any of ER's functions, which results in the disruption of the proper folding and secretory capacity of the ER and increased load of unfolded proteins in its lumen. ER stress activates a complex and multifaceted intracellular signal transduction pathway that is essentially designed to reestablish ER homeostasis. ER stress, is sensed by the ER chaperone protein glucose regulated protein 78 (GRP78) which in turn regulates three known ER resident proteins: inositol requiring protein-1 (IRE1), protein kinase RNA-like ER kinase (PERK), and activating transcription factor-6 (ATF6), and then converge to drive the expression of C/EBP homologous protein (CHOP).

As used herein, the term “ER stress inhibitor” refers to a natural or synthetic compound that has a biological effect to inhibit the activity or the expression of ER stress. More particularly, such compound is capable of inhibiting to ER homeostasis. More particularly, said inhibitor is able to inhibit ER resident proteins such as PERK.

Inventors have shown that the combination of ABT-263 or a derivative thereof with PERK inhibition synergistically reduced the survival rate of primary uveal melanoma cells.

In a particular embodiment, the ER stress inhibitor is a PERK inhibitor.

Accordingly, the invention relates to a i) compound ABT-263 or a derivative thereof, and ii) a PERK inhibitor, as a combined preparation for simultaneous, separate or sequential use in the treatment of primary uveal melanoma, metastatic uveal melanoma and/or uveal melanoma resistant.

As used herein, the term “PERK” also known as Eukaryotic translation initiation factor 2-alpha kinase 3 (EIF2AK3) refers to a protein kinase RNA-like ER kinase which is an enzyme that in humans is encoded by the EIF2AK3 gene.

In a particular embodiment, the PERK inhibitor is a natural or synthetic compound that has a biological effect to inhibit the activity or the expression of PERK. More particularly, the PERK inhibitor inhibits, reduces, or otherwise decreases the activity of or signaling through the Protein Kinase RNA-like.

In a particular embodiment, the PERK inhibitor is a peptide, petptidomimetic, small organic molecule, antibody, aptamers, siRNA or antisense oligonucleotide. PERK inhibitor s are disclosed in WO2015/056180 and WO2014/161808.

In a particular embodiment, the PERK inhibitor is small molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the PERK inhibitor is GSK2606414 a cell-permeable PERK inhibitor.

In a particular embodiment, the GSK2606414 has the following chemical formula and structure: CAS number: 1337531-36-8, C24H20F3N50:

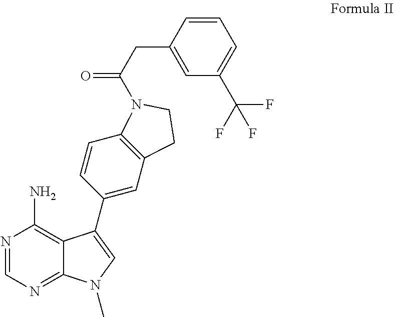 [SMILES: Cn1cc(-c2ccc3c(c2)CCN3C(=O)Cc2cccc(C(F)(F)F)c2)c2c(N)ncnc21]

In some embodiments, the PERK inhibitor is a short hairpin RNA (shRNA), a small interfering RNA (siRNA) or an antisense oligonucleotide which inhibits the expression of PERK. In a particular embodiment, the inhibitor of PERK expression is siRNA.

A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of PERK expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homology-directed repair (HDR).

In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

In a particular embodiment, the ER stress inhibitor is a CHOP inhibitor. As used herein, the term CHOP refers to C/EBP homologous protein. CHOP plays an important role in ER stress-induced apoptosis. CHOP belongs to the family of CCAAT/enhancer binding proteins (C/EBPs) and is involved in the regulation of genes that encode proteins involved in proliferation, differentiation and expression, and energy metabolism.

In a particular embodiment, the ER stress inhibitor is an IRE1α (referred to as IRE1 hereafter) inhibitor. IRE1α is a serine/threonine kinase and endoribonuclease that represents the most conserved UPR signaling branch in evolution, controlling cell fate under ER stress. This protein possesses intrinsic kinase activity and an endoribonuclease activity.

In a particular embodiment, the ER stress inhibitor is an inhibitor of RNase activity of IRE1.

As used herein, the term “RNase activity of IRE1” refers to the activity of the endoribonuclease domain of IRE1 which either degrades specific RNA (mRNA or microRNA) to avoid their translation or their cellular activity (in the case of microRNA), an activity known as the RIDD (regulated IRE1-dependent decay of RNA), or contributes to the splicing XBP1 (X-box-binding protein 1) mRNA to change the reading frame leading to the production of a novel protein (XBP1s), a potent unfolded-protein response transcriptional activator.

As used herein, the term “inhibitor of RNase activity of IRE1” has its general meaning in the art and refers to any compound, natural or synthetic, that blocks, suppresses, or reduces (including significantly) the RNase activity of IRE1. The term “inhibitor of RNase activity of IRE1” includes but is not limited to: small organic molecule, polypeptide, peptidomimetics.

In some embodiments, the RNase activity of IRE1 inhibitor is a small organic molecule.

In one embodiment, the inhibitor of the RNase activity of IRE1α is a RNase domain inhibitor.

In one embodiment, the inhibitor of the RNase activity of IRE1α is a kinase inhibitor.

In one embodiment, the inhibitor of the RNase activity of IRE1α is a type I kinase inhibitor. In one embodiment, the inhibitor of the RNase activity of IRE1α is type II kinase inhibitor.

In a particular embodiment, the inhibitor of the RNase activity of IRE1α is STF083010.

As used herein, the term “STF083010” has its general meaning in the art and refers to N-[(2-Hydroxy-1-naphthalenyl)methylene]-2-thiophenesulfonamide.

In one embodiment, the inhibitor of the RNase activity of IRE1α is 4μ8c.

In one embodiment, the inhibitor of the RNase activity of IRE1α is Irestatin.

In one embodiment, the inhibitor of the RNase activity of IRE1α is MG132.

In one embodiment, the inhibitor of the RNase activity of IRE1α is 17-AAG.

In one embodiment, the inhibitor of the RNase activity of IRE1α is 1-NM-PP1.

In one embodiment, the inhibitor of the RNase activity of IRE1α is Lactacystin.

In one embodiment, the inhibitor of the RNase activity of IRE1α is MKC-3946.

In one embodiment, the inhibitor of the RNase activity of IRE1α is toyocamycin.

In one embodiment, the inhibitor of the RNase activity of IRE1α is 3-methoxy-6-bromosalicylaldehyde.

In one embodiment, the inhibitor of the RNase activity of IRE1α is APY29.

In one embodiment, the inhibitor of the RNase activity of IRE1α is sunitinib.

In one embodiment, the inhibitor of the RNase activity of IRE1α is KIRA6.

In one embodiment, the inhibitor of the RNase activity of IRE1α is a 4-phenylbutyric acid analogue (Zhang H, Nakajima S, Kato H, Gu L, Yoshitomi T, Nagai K, et al. Selective, potent blockade of the IRE1 and ATF6 pathways by 4-phenylbutyric acid analogues. Br J Pharmacol. oct 2013; 170(4):822-34).

In some embodiments, the RNase activity of IRE1 inhibitor is a polypeptide or fragment thereof.

The term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups).

The polypeptides of the invention may be produced by any suitable means, as will be apparent to those of skill in the art. In order to produce sufficient amounts of polypeptides for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

The polypeptides of the invention and fragments thereof according to the invention can exhibit post-translational modifications, including, but not limited to glycosylations, (e.g., N-linked or O-linked glycosylations), myristylations, palmitylations, acetylations and phosphorylations (e.g., serine/threonine or tyrosine).

In some embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

In some embodiments, the RNase activity of IRE1 inhibitor is a pepetidomimetics.

As used herein the term “peptidomimetic” means a peptide-like molecule that has the activity of the peptide upon which it is structurally based. Such peptidomimetics include chemically modified peptides, peptide-like molecules containing non-naturally occurring amino acids, and peptoids, and have an activity such as selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff, John Wiley & Sons 1995), pages 803-861). Peptidomimetics may be designed in order to increase peptide stability, bioavailability, solubility, etc

In a further embodiment, i) ABT-263 or a derivative thereof ii) an endoplasmic reticulum (ER) stress inhibitor and iii) a classical treatment, as a combined preparation for simultaneous, separate or sequential use in the treatment of uveal melanoma.

In a further embodiment, i) ABT-263 or a derivative thereof, ii) an endoplasmic reticulum (ER) stress inhibitor and iii) a classical treatment, as a combined preparation for simultaneous, separate or sequential use in the treatment of primary uveal melanoma.

In a further embodiment, i) ABT-263 or a derivative thereof, ii) an endoplasmic reticulum (ER) stress inhibitor and iii) a classical treatment, as a combined preparation for simultaneous, separate or sequential use in the treatment of metastatic uveal melanoma and/or uveal melanoma resistant.

As used herein, the classical treatment refers to radiotherapy, protonotherapy, chemotherapy, immunotherapy and/or targeted therapy as defined above.

As used herein the terms “administering” or “administration” refer to the act of injecting or otherwise physically delivering a substance as it exists outside the body (e.g., ABT-263 and/or an ER stress inhibitor) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof. In a particular embodiment, the subject is administered subcutaneously, intradermally or topically with a compound which restores the activity of p63 (e.g., ABT-263 and/or an ER stress inhibitor).

In a further embodiment, the method according to the invention, wherein ABT-263 or a derivative thereof and/or an ER stress inhibitor compound are administered to the subject in need thereof simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In a particular embodiment, the compound ABT-263 or a derivative thereof according to the invention is administered by orally, systemically, intravitreously or topically.

In a particular embodiment, the compound ABT-263 or a derivative thereof according to the invention is administered by ophthalmic drop or an ophthalmic ointment.

In a particular embodiment, the combined preparation according to the invention, wherein said combined preparation is administered by orally, systemically, intravitreously or topically.

In a particular embodiment, the compound ABT-263 and/or an endoplasmic reticulum (ER) stress inhibitor are formulated for an intravitreous administration.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient (ABT-263 and/or an ER stress inhibitor) for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition

The compound ABT-263 or a derivative thereof and/or an ER stress inhibitor for use according to the invention alone and/or combined with classical treatment as described above may be combined with pharmaceutically acceptable excipients, and optionally sustained-release matrices, such as biodegradable polymers, to form pharmaceutical compositions.

Accordingly, in a third aspect, the invention relates to a pharmaceutical composition comprising a compound ABT-263 or a derivative thereof.

In a fourth aspect, the invention to a pharmaceutical composition comprising a i) compound ABT-263 or a derivative thereof, and ii) an ER stress inhibitor.

In some embodiments, the pharmaceutical composition according to the invention for use in the treatment of uveal melanoma, primary uveal melanoma, metastatic uveal melanoma and/or uveal melanoma resistant.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the pharmaceutical composition comprising compound ABT-263 or a derivative thereof and/or an endoplasmic reticulum (ER) stress inhibitor is formulated for an oral, systemic, intravitreous or topical administration.

In a particular embodiment, the pharmaceutical composition comprising compound ABT-263 or a derivative thereof and/or an endoplasmic reticulum (ER) stress inhibitor is formulated for an intravitreous administration.

The invention will be further illustrated by the following figures and examples. However, these examples and figures should not be interpreted in any way as limiting the scope of the present invention.

### FIGURES

FIG. 1. ABT-263 induces apoptosis of uveal melanoma cells. Detection of the mitochondrial membrane potential using TMRE staining in Mel270 and OMM1 uveal melanoma cells exposed to ABT-263 3 μM or 5 μM for 48 h in the absence or presence of QVD-OPh 20 μM. Representative FACS plot and quantification of TMRE positive cells are shown. ***P-value<0.001 of Bonferroni post hoc 2-way anova test.

FIG. 2. Effect of ABT-263 on tumor growth in vivo effects. (A) Primary Mel270 and (B) metastatic OMM1 uveal melanoma cells were subcutaneously engrafted into athymic nude mice. Once the tumours reached 100 mm3, mice were treated daily with ABT-263. The arrow indicates the start of the treatment. The growth tumor curves were determined by measuring the tumor volume using the equation V=(L Å˜W2)/2. Results are presented as mean (±SEM) tumor volumes (mm3) and *P-value<0.05, **P-value<0.01, ***P-value<0.001 are from Bonferroni post hoc 2-way anova independent variance test in ABT-263 treated versus vehicle at each point. (C-D) Sub-cutaneous tumor weight from control (Ct) and ABT-263-treated mice is shown.

FIG. 3. ABT-263 reduces tumor growth in vivo. (A) Primary 92.1 uveal melanoma cells were subcutaneously engrafted into athymic nude mice. Once the tumours reached 100 mm3, mice were treated daily with ABT-263. The arrow indicates the start of the treatment. The growth tumor curves were determined by measuring the tumor volume using the equation V=(L Å˜W2)/2. Results are presented as mean (±SEM) tumor volumes (mm3) and *P-value<0.05, **P-value<0.01 is from Bonferroni post hoc 2-way anova independent variance test in ABT-263 treated versus vehicle at each point. (B) Sub-cutaneous tumor weight from control (Ct) and ABT-263-treated mice is shown. (C) Blood biochemical analysis of mice xenografted with OMM1 cells. Serum activity of alanine transaminase (ALT) and aspartate transaminase (AST) was measured in control and ABT-263 by spectrophotometric methods after the end of treatment. Data are presented as mean±SD (n=5 per group).

FIG. 4: ABT-263 induces ER stress and activation of the unfolded protein response. Western blot of ER stress molecules in Mel270 cells treated with ABT-263 5 μM in the absence or presence of QVD-OPh 20 μM. Representative western blot are shown.

FIG. 5: PERK dampens ABT-263 killing activity. (A) Q-PCR analysis of PERK level in Mel270 uveal melanoma cells treated with control (siCtl) or PERK (siPERK) siRNA for 48 hr. (B) Mel270 cells were treated with control (siCtl) or PERK (siPERK) siRNA for 48 hrs before being exposed to ABT-263 3 μM for 48 hr. FACS analyses of Annexin V/DAPI double staining in cells freshly isolated from a human biopsy indicate alive (white) or dead (early, late apoptosis and necrosis in light grey) cells. (C) Mel270 cells were treated with ABT-263 5 μM or GSK2606414 5 μM alone or in combination for 48 h.

### EXAMPLE

Material & Methods

Cell Cultures and Reagents

Human uveal melanoma cell lines and short-term cultures derived from different patients with metastatic malignant melanoma cells were grown in DMEM supplemented with 7% FBS at 37° C. in a humidified atmosphere containing 5% CO2. Lipofectamine™ RNAiMAX and opti-MEM medium were purchased from Invitrogen (San Diego, CA, USA). ABT-263 was obtained from Euromedex and qVD from Clinisciences.

Western Blot Assays

Western blotting was performed as previously described 25,26. Briefly, cell lysates (30 μg) were separated using SDS-PAGE, transferred onto a PVDF membrane and subsequently exposed to the appropriate antibodies, anti-BCL2 (ms-123-PO) was from neomarker, anti-caspase 3 (#610323) was from BD, anti-BAX (#8429) was from sigma, anti-PARP (#9542), anti-BCL-XL (#2762), anti-caspase 9 (#9502) and anti-PUMA (#4976) were from Cell Signaling Technology Inc, anti-MCL1 (#sc-819), anti-NOXA (#56169), and anti-HSP90 (#sc-13119) were from Santa Cruz biotechnology. The proteins were visualized using the ECL system (Amersham). The western blots shown are representative of at least 3 independent experiments.

Cell Death Analysis by Flow Cytometry

Cells were seeded at a density of 100 000 cells/well, in 6-well plate and treated with ABT-263 for indicated time. Cells were harvested using accutase enzyme, washed twice with ice-cold phosphate-buffered saline, resuspended in a buffer (Hepes 250 mM, NaCl 150 mM, KCl 5 mM, MgCl2 2 mM, CaCl2) 2 mM) with DAPI (1 μg/ml) and Annexin V-Alexa Fluor 647 conjugate (1/100) and incubated for 15 minutes at room temperature (25° C.) in the dark. Samples were immediately analyzed by a flow cytometer (MACS QUANT) using a laser at 405 nm excitation with a bandpass filter at 425 nm and 475 nm for DAPI detection and a laser at 635 nm excitation with a bandpass filter at 650 nm and 665 nm for Alexa Fluor 647 dye. Annexin and DAPI mono- or double positive cells were counted as dead cells.

Colony Formation Assay

Human uveal melanoma cells were seeded onto 6-well plates. The cells were subsequently placed in a 37° C., 5% C02 incubator. Colonies of cells were grown before being stained with 0.04% crystal violet/2% ethanol in PBS for 30 min. Photographs of the stained colonies were captured. The colony formation assay was performed in duplicate.

mRNA Preparation and Real-Time/Quantitative PCR

The mRNA was isolated using TRIzol (Invitrogen) according to a standard procedure. QRT-PCR was performed using SYBR® Green I (Eurogentec, Seraing, Belgium) and Multiscribe Reverse Transcriptase (Applied Biosystems) and subsequently monitored using the ABI Prism 7900 Sequence Detection System (Applied Biosystems, Foster City, CA). The detection of the SB34 gene was used to normalize the results. Spliced Xbp1 primers were previously reported27. Primer sequences for each cDNA were designed using either Primer Express Software (Applied Biosystems) or qPrimer depot (http://primerdepot.nci.nih.gov), and these sequences are available upon request.

Animal Experimentation

Animal experiments were performed in accordance with French law and approved by a local institutional ethical committee. The animals were maintained in a temperature-controlled facility (22° C.) on a 12-h light/dark cycle and provided free access to food (standard laboratory chow diet from UAR, Epinay-S/Orge, France). Human Mel270 uvealmelanoma cells were subcutaneously inoculated into 8-week-old female, immune-deficient, athymic, nude FOXN1nu mice (Harlan Laboratory). When the tumors became palpable (0.1-0.2 cm3), the mice received an intraperitoneal injection of ABT-263 (50 mg/kg), dissolved in 10% DMSO 6 times per week. Control mice were injected with DMSO alone. The growth tumor curves were determined after measuring the tumor volume using the equation V=(L×W2)/2. At the end of the experiment, the mice were euthanized by cervical dislocation.

Statistical Analysis

The data are presented as the means±SD and analyzed using regular two-way ANOVA test and Bonferroni post-hoc tests with Graph Pad Prism. The difference between both conditions was statistically significant at p-value<0.05.

Results

ABT-263 Triggers Apoptotic Cell Death of Uveal Melanoma Cells.

Elevated expression of anti-apoptotic members of the BCL2 family in uveal melanoma cells compared to normal uveal melanocytes were previously reported^{5,14}. Hence, we conducted experiments to assess the effect of the ABT drugs, ABT-199, ABT-737 and ABT-263. Cell proliferation assay indicated that three out of the four cell lines assessed were not sensitive to ABT-199. Only OMM1 cells showed moderate proliferation inhibition (15%) in response to ABT-199 effect (data not shown). Although primary melanoma cells were also highly resistant to ABT-737 and ABT-263, both drugs strongly reduced proliferation of OMM¹ and OMM^{2,5} metastatic melanoma cells. Similar observations were performed when considering the cell death (data not shown).

Colony formation assay also demonstrated that metastatic cell lines were more sensitive to the long-term growth inhibitory activity of ABT-263 (data not shown).

Western blot indicated that ABT-263 effect was associated with cell cycle arrest in Mel270 cells, as shown by reduced CDK4 expression and increased in the cell cycle inhibitor p27 and p21 (data not shown). Since ABT-263 is in clinical trial, for subsequent studies, we decided to proceed with ABT-263, which proved to be a potent anti-metastatic melanoma strategy.

We next assessed the ability of ABT-263 to induce apoptosis in the panel of human uveal melanoma cell lines. ABT-263 cytotoxicity as measured by Annexin V/DAPI staining revealed that metastatic uveal melanoma cells were much more sensitive than the primary uveal melanoma cell lines, inducing 80% to 20-35% cell death respectively (data not shown). qVD resulted in all cell lines to have a protective effect. ABT-263 killing effect was also found in short-term cultures of primary uveal melanoma tumour (data not shown). Additionally, ABT-263 induced a time-dependent disappearance of full-length PARP protein, and caspase 3 zymogen (data not shown). The disappearance of total poly ADP-ribose polymerase (PARP) and zymogenic caspase 3 also revealed that the metastatic uveal melanoma cells were more sensitive to ABT-263 effect than the primary cells.

Comparison of ABT-263 effect in both primary and metastatic cells showed a dose-dependent cleavage of PARP, caspase 9 and caspase 3. Cleaved caspase 3 was detected earlier in the OMM¹ metastatic cells than in Mel270 cells (data not shown). As confirmation that cell death was due to apoptosis, pretreatment with the pan caspase inhibitor QVD prevented PARP and caspase cleavage (data not shown).

Protein immunoblot revealed that expression level of the main anti-apoptotic BCL-2 subfamily members (BCL2, MCL1, BCL-XL) was higher in the metastatic as compared with the primary cells (data not shown). After 24 h of treatment, the only noticeable change in primary cells was the increase in pro-apoptotic NOXA level (data not shown). In the metastatic OMM1 cells only, ABT-263 cytotoxic effect was accompanied by a reduced expression of pro-survival (BCL-xL, MCL1), and pro-apoptotic (PUMA, BAX) members (data not shown). BCL2 exhibited no change in response to ABT-263.

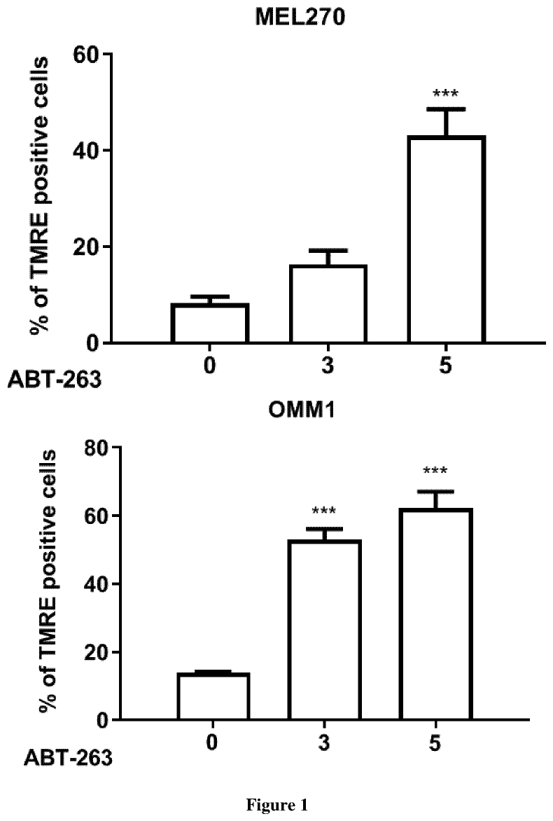

ABT-263 also dose-dependently reduced the mitochondrial membrane potential ΔΨm (FIG. 1). Interestingly, while qVD treatment prevented mitochondrial membrane depolarization observed following ABT-263 exposure in OMM¹ cells (data not shown), in Mel270 cells it did not prevent it (data not shown), suggesting different mechanisms of cell death induction operate in these the two cell lines.

Altogether, these data demonstrated that ABT-263 induced cell death via apoptosis in uveal melanoma cells, with the metastatic cells being more sensitive that the primary cells.

ABT-263 Prevents Growth of Human Uveal Melanoma Tumor Xenografts

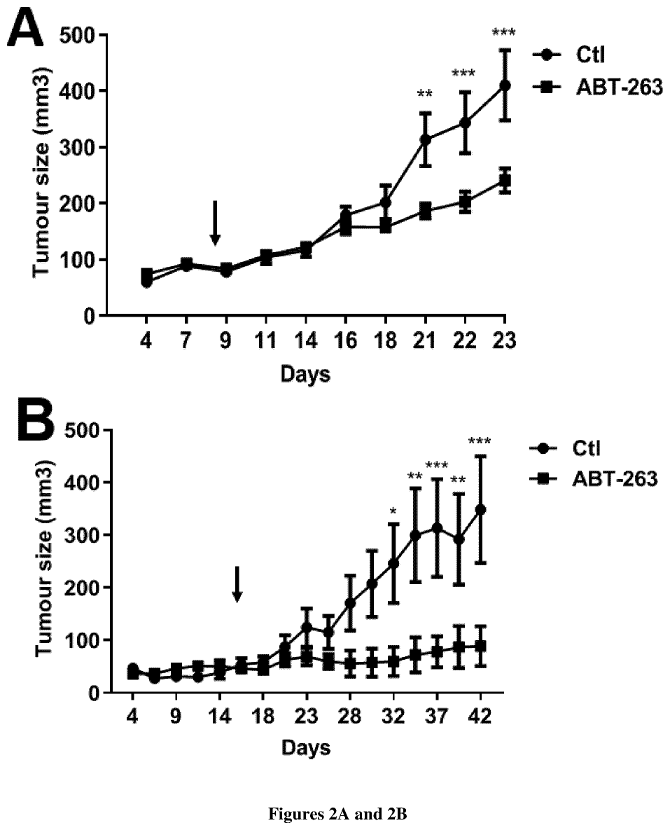

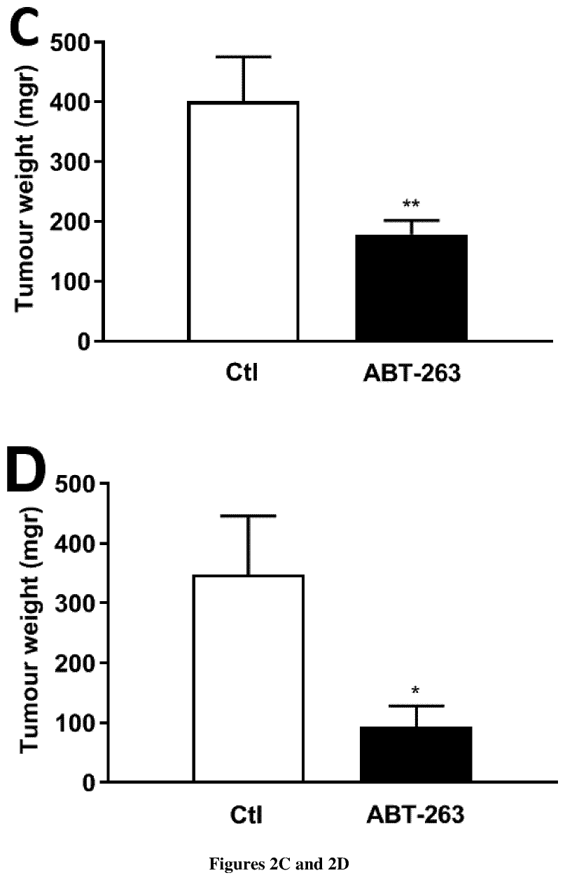

Experiments were next conducted in vivo to investigate the antineoplasic effect of ABT-263. To this aim, the primary Mel270, 92.1 and metastatic OMM1 human uveal melanoma cells were engrafted subcutaneously and treated with ABT-263 or with its vehicle (FIGS. 2A-2D).

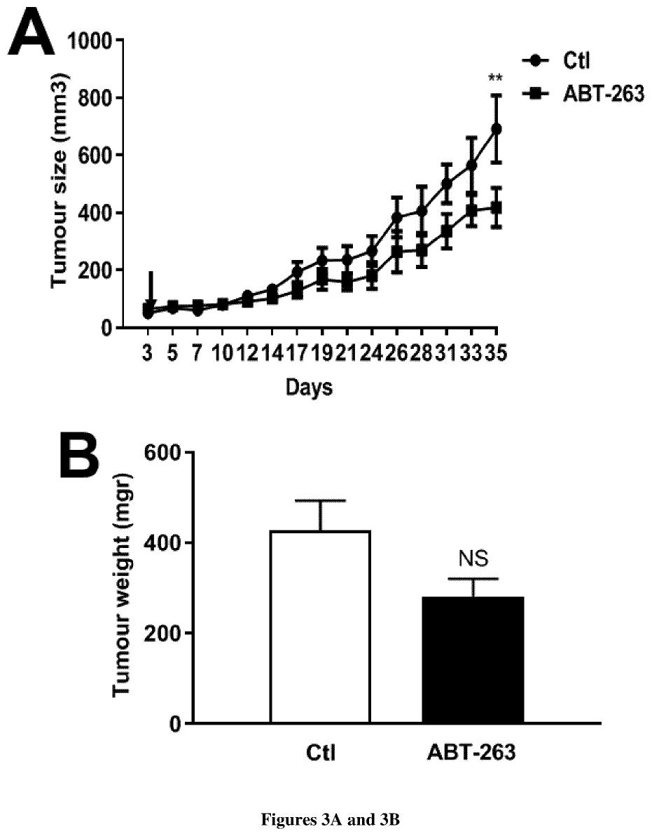

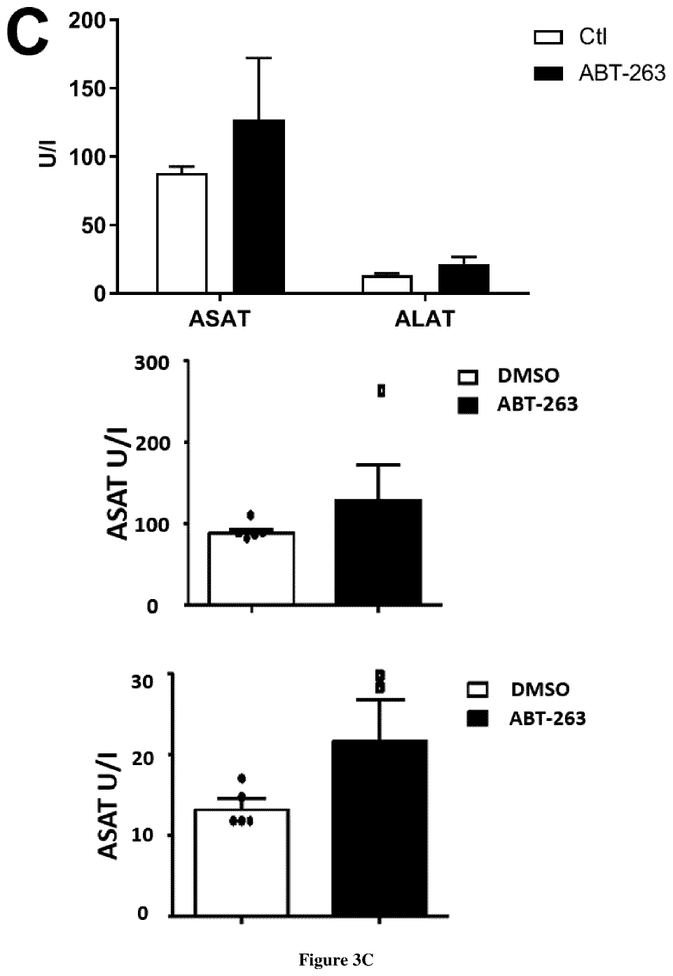

ABT-263 was found to be partially effective in established Mel270 xenografts (FIG. 3A) whereas it exhibited almost no effect on 92.1 cell growth (FIGS. 3A and 3B). Interestingly, ABT-263 induced marked tumor regression of metastatic OMM1 xenografts, leading in some mice to total tumor disappearance (FIG. 3B). Excised tumors in the ABT-263 group weighed significantly less than those in the control group (FIG. 3C). Furthermore, there was no significant differences in body weight between mice treated with vehicle or ABT-263 (data not shown), nor in serum alanine transaminase (ALT) and aspartate transaminase (AST) in OMM1-xenografted mice in both group (FIG. 3C), indicating that no hepatotoxicity was induced by ABT-263 under in vivo conditions. Thus, ABT-263 proved to be highly efficient in killing metastatic uveal melanoma cells and in preventing tumor growth also in vivo.

ABT-263 Induces Endoplasmic Reticulum (ER) Stress in Primary Uveal Melanoma Cells.

BCL-2 protein family is mainly known for its anti-apoptotic role operating at the mitochondria level, yet it is also recognized for its role in the endoplasmic reticulum (ER)¹⁵. Since our different cell lines present a different sensitivity to ABT-263, we decided to investigate in uveal melanoma cell lines perturbations of this pathway in response to ABT-263.

ER stress, is sensed by the ER chaperone protein glucose regulated protein 78 (GRP78/also called BIP) which in turn leads to the activation of three known ER resident proteins: inositol requiring protein-1 (IRE1), protein kinase RNA-like ER kinase (PERK), and activating transcription factor-6 (ATF6), and then converge to drive the expression of C/EBP homologous protein (CHOP)¹⁶.

ABT-263-mediated ER stress was analyzed in the primary and metastatic cell lines using detection of BIP, ATF4 and CHOP as surrogates for ER stress response. In both human Mel270 cells and 92.1 cells, ABT-263 enhanced ATF4 and CHOP level (data not shown). Contrastingly, induction of ATF4 and CHOP by ABT-263 was hardly detectable in the metastatic human uveal melanoma cells, and, BIP was not detected in OMM1 cells. We also did not detect PERK and IRE1α phosphorylation (Thy980 and Ser724 respectively) in metastatic cell lines treated with ABT-263 (data not shown).

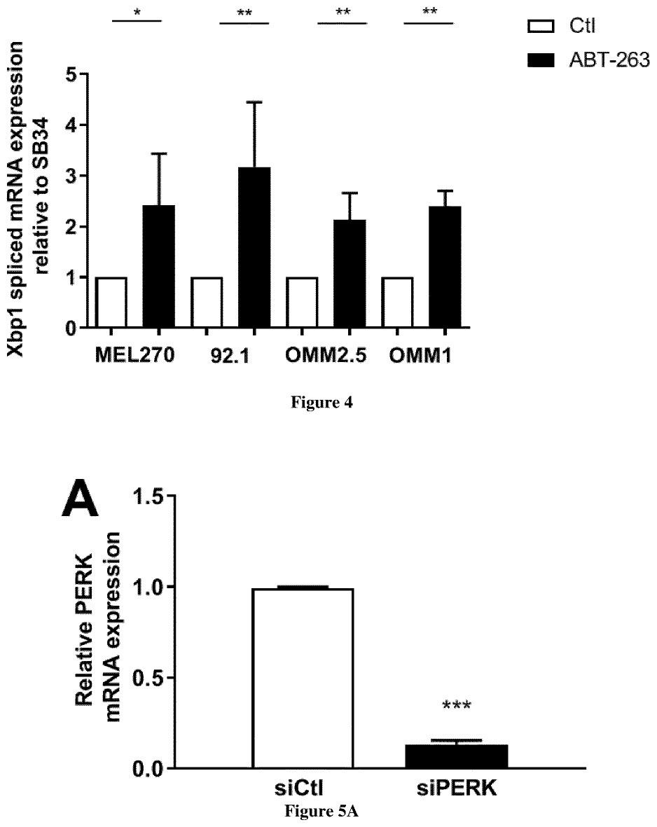

Of note, in contrast to ABT-263, tunicamycin, a known ER stress inducer was able to stimulate CHOP in OMM1, thus ruling out that the ER stress pathway is deficient in the metastatic cell lines (data not shown). Further, XBP1 splicing was significantly higher following ABT-263 exposure in both primary cells and in the metastatic OMM1 cells (FIG. 4).

Kinetics analysis in Mel270 primary uveal melanoma cells revealed that the protein level of BIP, was enhanced following treatment with ABT-263 (data not shown). PERK and EIF2α were phosphorylated (Thy980, Ser51 respectively) and ATF4 increased. We also observed IRE1α phosphorylation, XBP1 splicing and ATF6 rise and cleavage. There was also a significant upregulation of the very downstream ER stress effector CHOP (data not shown). Treatment with the pan caspase inhibitor qVD did not impact ABT-263 mediated change in ER molecules. These data indicate that in Mel270 primary uveal melanoma cells, ABT-263 activates all branches of the ER stress pathways, which operate upstream of the apoptotic pathway.

Altogether, our data indicate that ABT-263 elicits activation of ER stress in primary uveal melanoma cells, while there are signs of defective activation of this pathway in the metastatic cells.

ER Stress Protects Primary Uveal Melanoma Cells from ABT-263-Induced Apoptosis

Chemotherapy regimens were previously found to cause endoplasmic reticulum (ER) stress and to activate the unfolded protein response (UPR), which paradoxically is a process that keep the cancer cell away from apoptosis to promote their survival.

To better understand the role of ER stress activation on ABT-263 treatment in primary uveal melanoma cells, we used genetic and/or pharmacological interventions, focusing on PERK and IRE1α, which are the two main branches considered to elicit the pro-survival output.

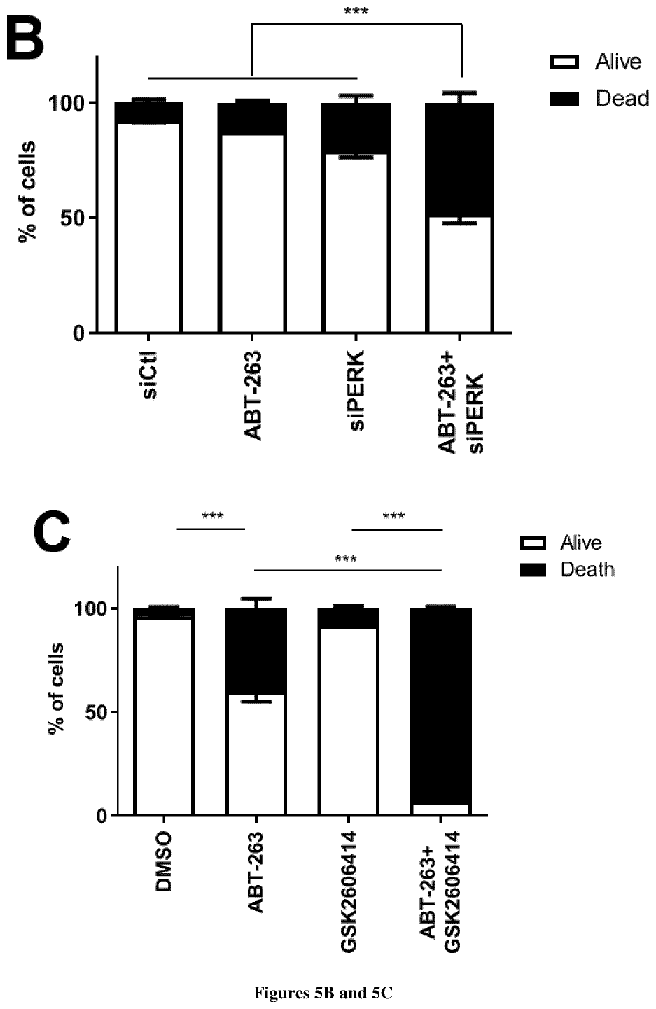

We first used siRNA to IRE1α. Whereas it efficiently reduced IRE1α expression in the two primary cell lines (Mel270 and 92.1), it did not enhance cell death mediated by ABT-263 treatment (data not shown). Further, although they are more sensitive to ABT-263, no additive effect was observed when combining ABT-263 with siRNA to IRE1α in the metastatic cells (data not shown). siRNA to PERK efficiently inhibited PERK expression (FIG. 5A) and activation as revealed by EIF2α loss of phosphorylation in both basal and ABT-263-treated cells (data not shown). To reveal the effect of combined ABT-263 and PERK down-regulation, we decreased ABT-263 concentration to 3 μM, which was shown to do not induce cell death on Mel270 (data not shown). Whereas ABT-263 treatment or PERK inhibition exhibited a relative low effect on PARP cleavage and on alive cell percentage (FIG. 5B) when used alone, the combination significantly improved ABT-263 effects and its killing efficacy. Interestingly, in 92.1 cells that were much more resistant to ABT-263-induced apoptosis, PERK knockdown sensitized these cells to ABT-263 effect at similar level as Mel270 (data not shown). We also tested the effect of GSK2606414, a cell-permeable PERK inhibitor. GSK2606414 inhibited CHOP and ATF4 accumulation demonstrating its efficacy (data not shown). Whereas GSK2606414 alone displayed no effect on viable cells, ABT-263 effect was improved when combined with GSK2606414 (FIG. 5C), consistent with data obtained with the siRNA. GSK2606414 also resensitized the 92.1 cells to ABT-263 (data not shown). Altogether, inhibition of anti-apoptotic BCL-2 proteins by ABT-263 induces a protective feedback response in primary uveal melanoma cells, via induction of the ER stress response that can be prevented with PERK inhibitor.

Accordingly, the inhibition of anti-apoptotic BCL-2 proteins by ABT-263 alone or in combination with ER stress inhibitor represents a valid therapeutic strategy for the treatment of uveal metastatic melanoma cells.

### REFERENCES

Throughout this application, various references describe the state of the art to which this invention pertains. The disclosures of these references are hereby incorporated by reference into the present disclosure.
1. Krantz, B. A., Dave, N., Komatsubara, K. M., Marr, B. P. & Carvajal, R. D. Uveal melanoma: epidemiology, etiology, and treatment of primary disease. Clin Ophthalmol 11, 279-289 (2017).
2. Singh, A. D., Bergman, L. & Seregard, S. Uveal melanoma: epidemiologic aspects. Ophthalmol Clin North Am 18, 75-84, viii (2005).
3. Rietschel, P. et al. Variates of survival in metastatic uveal melanoma. J Clin Oncol 23, 8076-8080 (2005).
4. Carvajal, R. D. et al. Metastatic disease from uveal melanoma: treatment options and future prospects. Br J Ophthalmol 101, 38-44 (2017).
5. Hussein, M. R. Analysis of Bcl-2 protein expression in choroidal melanomas. J Clin Pathol 58, 486-489 (2005).
6. Adams, J. M. & Cory, S. The BCL-2 arbiters of apoptosis and their growing role as cancer targets. Cell Death Differ. 25, 27-36 (2018).
7. Jay, V., Yi, Q., Hunter, W. S. & Zielenska, M. Expression of bcl-2 in uveal malignant melanoma. Arch Pathol Lab Med 120, 497-498 (1996).
8. Harb, J. G. et al. Bcl-xL anti-apoptotic network is dispensable for development and maintenance of CML but is required for disease progression where it represents a new therapeutic target. Leukemia 27, 1996-2005 (2013).
9. Tse, C. et al. ABT-263: a potent and orally bioavailable Bcl-2 family inhibitor. Cancer Res 68, 3421-3428 (2008).
10. Merino, D. et al. BH3-Mimetic Drugs: Blazing the Trail for New Cancer Medicines. Cancer Cell 34, 879-891 (2018).
11. Cleary, J. M. et al. A phase I clinical trial of navitoclax, a targeted high-affinity Bcl-2 family inhibitor, in combination with gemcitabine in patients with solid tumors. Invest. New Drugs 32, 937-945 (2014).
12. Vlahovic, G. et al. A phase I safety and pharmacokinetic study of ABT-263 in combination with carboplatin/paclitaxel in the treatment of patients with solid tumors. Invest. New Drugs 32, 976-984 (2014).
13. Greenman, C. et al. Patterns of somatic mutation in human cancer genomes. Nature 446, 153-158 (2007).
14. Némati, F. et al. Targeting Bcl-2/Bcl-×L induces antitumor activity in uveal melanoma patient-derived xenografts. PLoS One 9, 1-9 (2014).
15. Bonneau, B., Prudent, J., Popgeorgiev, N. & Gillet, G. Non-apoptotic roles of Bcl-2 family: The calcium connection. Biochim. Biophys. Acta—Mol. Cell Res. 1833, 1755-1765 (2013).
16. Bahar, E., Kim, J. Y. & Yoon, H. Chemotherapy resistance explained through endoplasmic reticulum stress-dependent signaling. Cancers (Basel). 11, 1-20 (2019).
17. Sulkowska, M., Famulski, W., Bakunowicz-Lazarczyk, A., Chyczewski, L. & Sulkowski, S. Bcl-2 Expression in primary uveal melanoma. Tumori 87, 54-57 (2001).
18. Nemati, F. et al. Targeting Bcl-2/Bcl-XL induces antitumor activity in uveal melanoma patient-derived xenografts. PLoS One 9, e80836 (2014).
19. Monni, O. et al. BCL2 Overexpression Associated With Chromosomal Amplification in Diffuse Large B-Cell Lymphoma. Blood 90, 1168-1174 (1997).
20. Yan, D. et al. Role of microRNA-182 in posterior uveal melanoma: regulation of tumor development through MITF, BCL2 and cyclin D2. PLoS One 7, e40967 (2012).
21. Song, E. et al. Zeaxanthin Induces Apoptosis in Human Uveal Melanoma Cells through Bcl-2 Family Proteins and Intrinsic Apoptosis Pathway. Evidence-Based Complement. Altern. Med. 2013, 1-12 (2013).
22. Rodriguez, D., Rojas-Rivera, D. & Hetz, C. Integrating stress signals at the endoplasmic reticulum: The BCL-2 protein family rheostat. Biochim. Biophys. Acta—Mol. Cell Res. 1813, 564-574 (2011).
23. Rozpedek, W. et al. The Role of the PERK/eIF2α/ATF4/CHOP Signaling Pathway in Tumor Progression During Endoplasmic Reticulum Stress. Curr. Mol. Med. 16, 533-544 (2016).
24. Shi, W. et al. Unravel the molecular mechanism of XBP1 in regulating the biology of cancer cells. J. Cancer 10, 2035-2046 (2019).
25. Hilmi, C. et al. IGF1 promotes resistance to apoptosis in melanoma cells through an increased expression of BCL2, BCL-X(L), and survivin. J. Invest. Dermatol. 128, (2008).
26. Bertolotto, C. et al. A SUMOylation-defective MITF germline mutation predisposes to melanoma and renal carcinoma. Nature 480, (2011).
27. Van Schadewijk, A., Van&apos; T Wout, E. F. A., Stolk, J. & Hiemstra, P. S. A quantitative method for detection of spliced X-box binding protein-1 (XBP1) mRNA as a measure of endoplasmic reticulum (ER) stress. Cell Stress Chaperones 17, 275-279 (2012).

1. A method of treating uveal melanoma in a subject in need thereof, comprising
administering to the subject a therapeutically effective amount of ABT-263 or a derivative thereof, wherein the ABT-263 has the formula

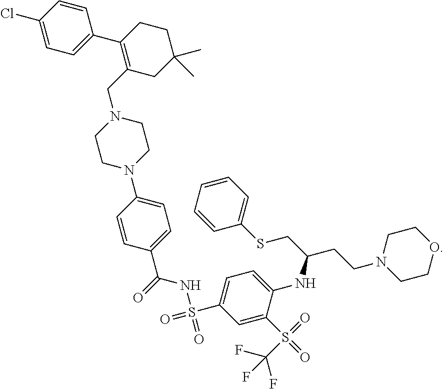 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

2. The method according to claim 1, wherein the uveal melanoma is metastatic uveal melanoma or resistant uveal melanoma.

3. The method according to claim to 2, wherein the resistant uveal melanoma is resistant to at least one conventional therapy selected from the group consisting of radiotherapy, chemotherapy, protonotherapy, targeted treatment and immunotherapy.

4. The method according to claim 1, wherein said compound is administered orally, systemically, intravitreously or topically.

5. A method of treating uveal melanoma, metastatic uveal melanoma and/or resistant uveal melanoma in a subject in need thereof, comprising,
administering to the subject a therapeutically effective amount of a combined preparation comprising compound ABT-263 or a derivative thereof, and ii) an endoplasmic reticulum (ER) stress inhibitor, wherein the compound ABT-263 has the formula

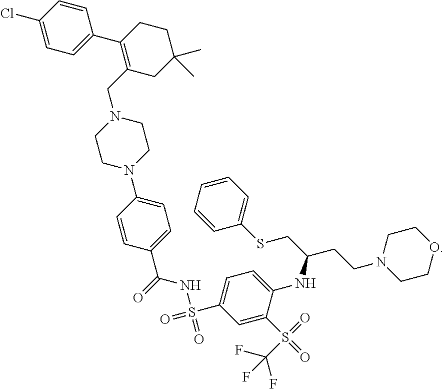 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

6. The method according to claim 5, wherein said ER stress inhibitor is a PERK, CHOP or IRE1α inhibitor.

7. The method according to claim 5, wherein said combined preparation is administered orally, systemically or intravitreously.

8. The method according to claim 5, wherein the ER stress inhibitor is a PERK inhibitor.

9. The method according to claim 8, wherein the PERK inhibitor is GSK2606414.

10. The method according to claim 8, wherein the PERK inhibitor is a siRNA.

11. (canceled)

12. A pharmaceutical composition comprising a i) compound ABT-263 or a derivative thereof, and ii) an ER stress inhibitor.

13. (canceled)

14. (canceled)

15. The method of claim 1, further comprising administering to the subject an ER stress inhibitor.

16. The method of claim 15, wherein the ER stress inhibitor is administered simultaneously, separately, sequentially or as a combined preparation with the ABT-263 or the derivative thereof.
